(19) United States
(12) Patent Application Publication
(10) Pub. No.: US 2022/0257251 A1
(43) Pub. Date: Aug. 18, 2022

(54) MAGNETIC COMPRESSION ANASTOMOSIS SYSTEM FOR LAPAROSCOPIC CHOLANGIOENTEROSTOMY

(71) Applicant: XI'AN MAGNETIC MEDICAL TECHNOLOGY CO., LTD, Xi'an (CN)

(72) Inventors: Yi LV, Xi'an (CN); Bo TANG, Xi'an (CN); Dinghui TANG, Xi'an (CN); Aihua SHI, Xi'an (CN)

(21) Appl. No.: 17/627,855

(22) PCT Filed: Sep. 25, 2019
(86) PCT No.: PCT/CN2019/107644

§ 371 Date: Jan. 18, 2022

(30) Foreign Application Priority Data
Aug. 9, 2019 (CN) 201910736134.2

(51) Int. Cl. A61B 17/11

(52) U.S. Cl. CPC A61B 17/11; A61B 2017/00876; A61B 2017/1125

(57) ABSTRACT

Disclosed is a magnetic compression anastomosis system for laparoscopic cholangioenterostomy, including an emission gun, daughter and parent magnetic rings, and an assisting rod, wherein the parent magnetic ring is positioned at the stump of a common bile duct in a purse string suture manner, the relative position is fixed by the assisting rod, the emission gun is loaded with the daughter magnetic ring and makes the same in butt joint with the parent magnetic ring by an intestinal cavity channel of an intestinal tube, the distance between the daughter magnetic ring and the parent magnetic ring is continuously shortened by a push rotary button of the emission gun, and when the relative distance between the daughter and the parent magnetic rings is shortest and mutual attraction is realized, separating the daughter magnetic ring from the emission gun is achieved by an activation mechanism of the emission gun, realizing laparoscopic cholangioenterostomy.

### TECHNICAL FIELD

The present application relates to the technical field of medical instrument, and in particular to an anastomosis system for laparoscopic cholangioenterostomy, and further in particular, to a magnetic compression anastomosis system for laparoscopic cholangioenterostomy.

### BACKGROUND ART

Minimal invasion is a trend in the development of surgery. Laparoscopy technology is an important way to achieve minimally invasive abdominal surgical operation. At present, many classic operations, such as cholecystectomy and subtotal gastrectomy, etc., of abdominal surgery can be made through laparoscopic methods. Due to the difficulty of laparoscopic anastomosis operation, some operations are still very difficult to implement under laparoscopic conditions, such as laparoscopic cholangioenterostomy. Magnetic compression anastomosis technology is a new surgical anastomosis technology using no suture line, with the outstanding advantage lying in the use of magnet interactions, such that it can achieve the continuous recanalization of channels without the traditional suture operations, which can greatly reduce the difficulty of the related suture operations under laparoscopic conditions.

At present, the devices used in laparoscopic magnetic compression cholangioenterostomy have appeared, which have significant advantages over the traditional manual sutures. However, the following problems still exist.

1) In the aspect of accuracy: previously, the laparoscopic magnetic compression cholangioenterostomy device can only be equipped with the daughter magnetic ring of one specification, wherein as for different tube diameters, it is needed to use an emission gun of different specifications, so that it cannot be flexibly adjusted according to the actual situation during the operation, wherein if the diameter is too small, it will cause the biliary stricture, and if the diameter is too large, it will cause the bile leakage.

2) In the aspect of operability: previously, when performing laparoscopic magnetic compression cholangioenterostomy, the most difficult operation is to make the emission gun and the parent magnetic ring be in butt joint with each other, wherein after the parent magnetic ring is inserted into the tail end of the bile duct through the purse string suture, its position cannot be fixed, wherein on the one hand, it will be retracted into the bile duct due to operating factors, and the radial relationship between the parent magnetic ring and the bile duct, after the parent magnetic ring retreats into the bile duct, often changes and at this time, the purse string suture must be performed again, and on the other hand, the butt-joint rod of the emission gun is not easy to enter the inner drainage tube of the parent magnetic ring, making it difficult to realize the butt joint.

3) In the aspect of safety: previously, the laparoscopic magnetic compression cholangioenterostomy device lacks a safety mechanism design, which cannot clarify the relative position of the daughter and parent magnetic rings, and cannot provide feedback on the implementation of key actions, which is not beneficial to the control on the operation process and in addition, no effective mechanism prevents the mis-operations.

Therefore, it is necessary to develop a magnetic compression anastomosis system for laparoscopic cholangioenterostomy that can reduce the difficulty of laparoscopic cholangioenterostomy and improve the accuracy and safety of laparoscopic cholangioenterostomy.

### SUMMARY

In view of the above problems, the present application provides a magnetic compression anastomosis system for laparoscopic cholangioenterostomy that can reduce the difficulty of laparoscopic cholangioenterostomy and improve the accuracy and safety of laparoscopic cholangioenterostomy.

For this purpose, the present application provides a magnetic compression anastomosis system for laparoscopic cholangioenterostomy, which comprises an emission gun, a daughter magnetic ring, a parent magnetic ring and an assisting rod, wherein the parent magnetic ring is positioned at a stump of a common bile duct in a purse string suture manner, and a relative position is fixed by means of the assisting rod, and the emission gun is loaded with the daughter magnetic ring and makes the daughter magnetic ring be in butt joint with the parent magnetic ring by means of an intestinal cavity channel of an intestine tube, and the assisting rod assists the parent magnetic ring to be in butt joint with the emission gun, and a distance between the daughter magnetic ring and the parent magnetic ring is continuously shortened by means of a push rotary button of the emission gun, wherein when a relative distance between the daughter magnetic ring and the parent magnetic ring is shortest and mutual attraction is achieved, separation of the daughter magnetic ring from the emission gun is achieved by means of an activation mechanism (excitation mechanism) of the emission gun, and the laparoscopic cholangioenterostomy is realized.

Optionally, the emission gun comprises a push rotary button, a butt-joint rod, an activation sleeve (excitation sleeve), a loading sleeve, a loading slot, a activation mechanism, a housing, a sensory feedback mechanism and a striker (firing pin), wherein a front end of the push button is connected to a rear end of the butt-joint rod through a spring mechanism, a front end of the butt-joint rod is provided with a hook structure that can be in butt joint with the parent magnetic ring, the butt-joint rod is located in the activation sleeve, the activation sleeve is connected to the activation mechanism, the sensory feedback mechanism is fixed on the activation sleeve, and the striker is arranged in the housing, wherein when the activation sleeve moves forward, the sensory feedback mechanism can contact the striker, the activation sleeve is located in the loading sleeve, the loading sleeve is fixed on the housing, a front end of the loading sleeve is detachably connected to a tail part of the loading slot, and the daughter magnetic ring is placed in the loading slot.

Optionally, the emission gun further comprises a display pin and a display frame, the display pin is fixed on the butt-joint rod, and the display frame is arranged in the housing and configured to display a position of the display pin.

Optionally, the emission gun further comprises a safety bolt, and the activation sleeve is connected to the safety bolt.

Optionally, the push rotary button, the loading slot, the safety bolt, the activation mechanism, and the housing, the sensory feedback mechanism, the display pin and the display frame are made of polymer material, and the butt-joint rod, the activation sleeve, the loading sleeve and the striker are made of non-paramagnetic stainless steel material.

Optionally, the daughter magnetic ring is ring-shaped, made of N50 neodymium iron boron (NdFeB) material, and coated with titanium nitride (TiN) on a surface thereof, and has an inner diameter of 5 mm and an outer diameter of 8-15 mm.

Optionally, the parent magnetic ring is composed of a parent magnetic casing, a parent magnet and an inner drainage tube, wherein the parent magnet is loaded in the parent magnet casing, the parent magnet casing is fixedly connected with the inner drainage tube, and the inner drainage tube is provided therein with a slot structure, and the slot structure can be in butt joint with the hook structure at the front end of the butt-joint rod.

Optionally, both the parent magnetic casing and the inner drainage tube are made of non-paramagnetic stainless steel material and have an inner diameter of 3 mm, an outer diameter of the parent magnetic casing is 8-15 mm, an outer diameter of the inner drainage tube is 4 mm, and the parent magnet is ring-shaped and made of N50 neodymium iron boron material, and is coated with titanium nitride on a surface thereof, and has an inner diameter of 5 mm and an outer diameter of 7.5-14.5 mm.

Optionally, the assisting rod consists of a supporting mechanism, a triggering mechanism, a connecting rod, a movable joint, and a clamping mechanism, wherein the supporting mechanism provides support for the triggering mechanism and the clamping mechanism, and the triggering mechanism is connected to the supporting mechanism through a first rotating shaft, so that the triggering mechanism can be open and closed relative to the supporting mechanism, around the first rotating shaft, and a rear end of the connecting rod is connected to the triggering mechanism and a front end thereof is connected to a rear end of the movable joint through a second rotating shaft, and a front end of the movable joint is connected to the clamping mechanism through a third rotating shaft, wherein the triggering mechanism will drive, when being opened relative to the supporting mechanism around the first rotating shaft, the connecting rod to move in a direction toward the clamping mechanism and realizes through the movable joint the opening of the clamping mechanism, and correspondingly, the triggering mechanism will drive, when being closed relative to the supporting mechanism around the first rotating shaft, the connecting rod to move in a direction toward the supporting mechanism, and realize the closing of the clamping mechanism through the movable joint, and the clamping mechanism can clamp the inner drainage tube of the parent magnetic ring, thereby fixing the parent magnetic ring at a relative position of the stump of the common bile duct and assisting the parent magnetic ring to be in butt joint with the emission gun.

Optionally, the supporting mechanism and the triggering mechanism are made of polymer material, and the connecting rod, the movable joint and the clamping mechanism (405) are made of non-paramagnetic stainless steel.

Compared with the prior art, the magnetic compression anastomosis system for laparoscopic cholangioenterostomy of the present application has the following beneficial effects:

1. improving the accuracy of laparoscopic magnetic compression cholangioenterostomy, wherein the combination of the loading slot and the loading sleeve in the present application is a detachable design, which allows to select the daughter magnetic ring of an appropriate specification according to the actual situation in the operation, and one design of the emission gun can meet requirements of a variety of application scenarios, improving the accuracy of the operation and reducing the cost of the patient at the same time;

2. reducing the operation difficulty of laparoscopic magnetic compression cholangioenterostomy, wherein the assisting rod is additionally provided in the present application, with the clamping mechanism thereof capable of effectively fixing the relative position of the parent magnetic ring, so as to prevent it from retracting into the bile duct and avoid performing the purse string suture again, which is also easy to realize the butt joint with the emission gun; and

3. improving the safety of laparoscopic magnetic compression cholangioenterostomy, wherein the display pin is combined with the display frame in the present application, which can display the relative positions of the daughter magnetic ring and the parent magnetic ring, and can assist the excitation operation; the sensory feedback mechanism will operate with the striker, so as to produce a “click” sound, by which the operator feels a sense of breakthrough, and a feedback on the completion of the excitation action is given; and the safety bolt can limit the movement of the activation mechanism, avoiding the accidental excitation of the daughter magnetic ring, and reducing the occurrence of adverse events.

### BRIEF DESCRIPTION OF DRAWINGS

FIG. 1 is a schematic diagram of the structure and application of the magnetic compression anastomosis system for laparoscopic cholangioenterostomy of the present application;

FIG. 2 is a schematic structural diagram of the emission gun of the magnetic compression anastomosis system for laparoscopic cholangioenterostomy of the present application;

FIG. 3 is an enlarged exploded view of the part D in FIG. 2;

FIG. 4 is a schematic structural diagram of the daughter magnetic ring of the magnetic compression anastomosis system for laparoscopic cholangioenterostomy of the present application;

FIG. 5 is a schematic structural diagram of the parent magnetic ring of the magnetic compression anastomosis system for laparoscopic cholangioenterostomy of the present application;

FIG. 6 is a schematic structural diagram of the assisting rod of the magnetic compression anastomosis system for laparoscopic cholangioenterostomy of the present application;

FIG. 7 shows a schematic diagram when the daughter magnetic ring is installed in the loading slot;

FIG. 8 shows a schematic diagram in which the hook structure at the front end of the butt-joint rod and the slot structure in the inner drainage tube of the parent magnetic ring are in butt joint with each other;

FIG. 9 shows a schematic diagram in which the activation sleeve pushes the daughter magnetic ring to be disengaged from the loading slot and makes the parent magnetic ring separated from the emission gun.

### DETAILED DESCRIPTION OF EMBODIMENTS

In order to make the objectives, technical solutions, and advantages of the embodiments of the present application clearer, the technical solutions in the embodiments of the present application will be clearly described as follows, with reference to the drawings in the embodiments of the present application. Obviously, part, but not all, of the embodiments of the present application are described.

It should be noted that, herein, the words “first”, “second”, “third”, and “fourth” are only intended to distinguish same names, rather than implying the relationship or order among these names.

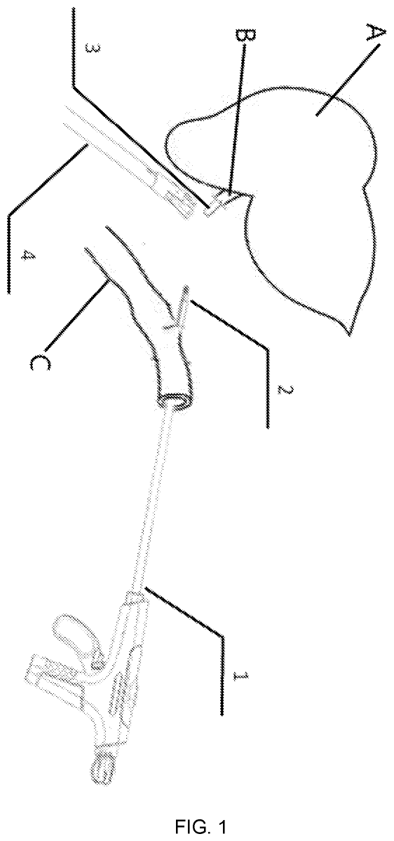

The present application relates to a magnetic compression anastomosis system for laparoscopic cholangioenterostomy. As shown in FIG. 1, it is composed of an emission gun 1, a daughter magnetic ring 2, a parent magnetic ring 3 and an assisting rod 4.

Herein, the parent magnetic ring 3 is positioned at the stump of the common bile duct B connected to the liver A, in a purse string suture manner, and the relative position is fixed by the assisting rod 4. The emission gun 1 is loaded with the daughter magnetic ring 2 and the daughter magnetic ring 2 is made to be in butt joint with the parent magnetic ring 2 through the intestinal cavity channel of the intestinal tube C. The assisting rod 4 assists the parent magnetic ring 3 to be in butt joint (docking) with the emission gun 1. After the butt joint, the assisting rod 4 is withdrawn, and the distance between the daughter magnetic ring 2 and the parent magnetic rings 3 is continuously shortened through the push rotary button 101 of the emission gun 1. When the relative distance between the daughter magnetic ring 2 and the parent magnetic ring 3 is shortest and mutual attraction is achieved, the emission gun 1 is separated from the daughter magnetic ring 2 through the activation mechanism 107 of the emission gun 1 to achieve the laparoscopic cholangioenterostomy.

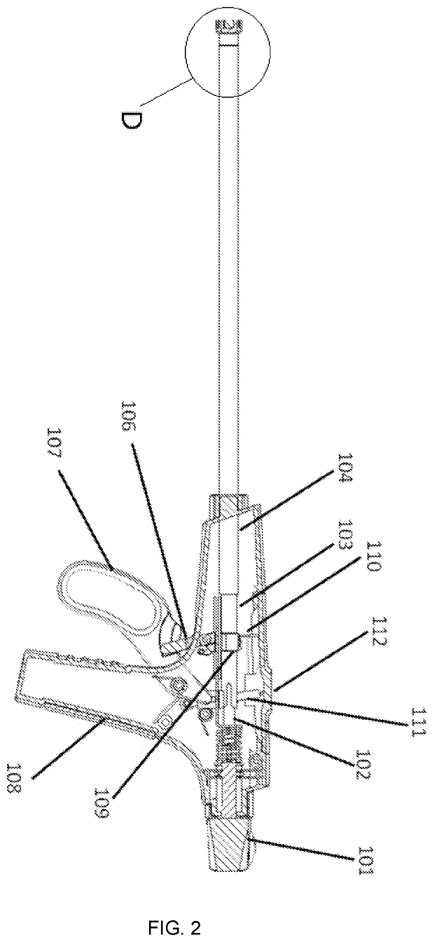

As shown in FIG. 2, the emission gun 1 comprises a push rotary button 101, a butt-joint rod 102, a activation sleeve 103, a loading sleeve 104, a loading slot 105, a activation mechanism 107, a housing 108, a sensory feedback mechanism 109, and a striker 110.

Herein, the housing 108 is used to provide support for other mechanisms of the emission gun 1, as long as it comprises a handle and a buttstock structure, with its specific structure not limited.

The front end of the push button 101 and the rear end of the butt-joint rod 102 are connected with each other by a spring mechanism. The push rotary button 101 and the rear end of the housing 108 are connected with each other by threads. In this way, by rotating the push rotary button 101, the push rotary button 101 is made to be able to move forward or backward relative to the housing 108, thereby driving the butt-joint rod 102 to move forward or backward. Specifically, when the push rotary button 101 is turned clockwise, the push rotary button 101 is made to move forward relative to the housing 108, compresses the spring mechanism, and pushes the butt-joint rod 102 to move forward through the spring mechanism. When the push rotary button 101 is turned counterclockwise, the push rotary button 101 is made to move backward with respect to the housing 108, releases the compression of the spring mechanism so that the spring mechanism retracts, and drives the butt-joint rod 102 to move backward by means of the retraction of the spring mechanism.

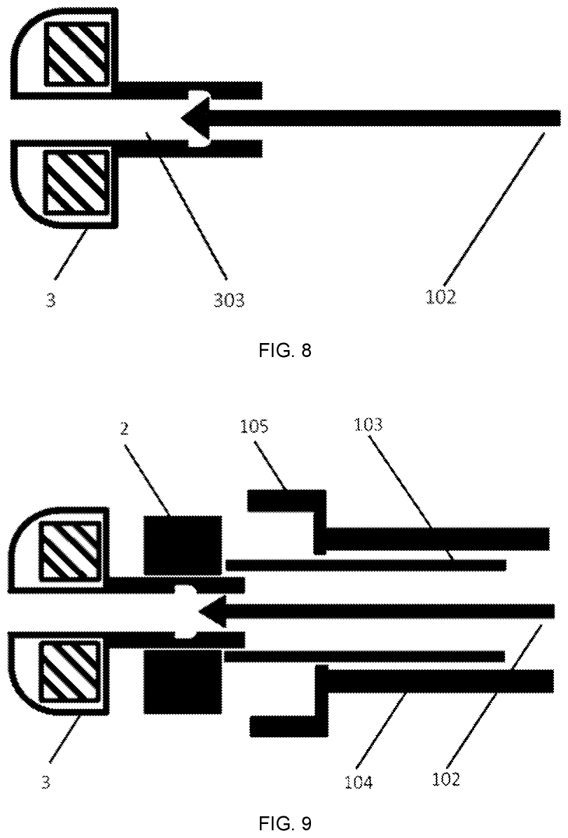

As shown in FIG. 8, the front end of the butt-joint rod 102 is provided with a hook structure capable of being in butt joint with the parent magnetic ring 3. Through the hook structure, the butt-joint rod 102 is made to be in butt joint with the parent magnetic ring 3.

Continuing to refer to FIG. 2, the butt-joint rod 102 is located in the activation sleeve 103. The activation sleeve 103 is connected to the activation mechanism 107. A person manually pulls the activation mechanism 107, and the activation sleeve 103 can be excited (driven) by the activation mechanism 107, so that the activation sleeve 103 moves forward. The principle of the activation mechanism 107 is the same as that of various existing pistol tools, which will not be described in detail here.

The sensory feedback mechanism 109 is fixed on the activation sleeve 103. The striker 110 is arranged in the housing 108. When the activation sleeve 103 moves forward, the sensory feedback mechanism 109 is driven to move forward, so that the sensory feedback mechanism 109 can contact the striker 110. In this way, the sensory feedback mechanism 109 will cooperate with the striker 110, to produce a “click” sound, by which the operator is made to feel a sense of breakthrough, which can give feedback on the completion of the excitation action, so that the operator knows whether the operation is completed, thereby improving safety of the operation.

The activation sleeve 103 is located in the loading sleeve 104. The loading sleeve 104 is fixed on the housing 108.

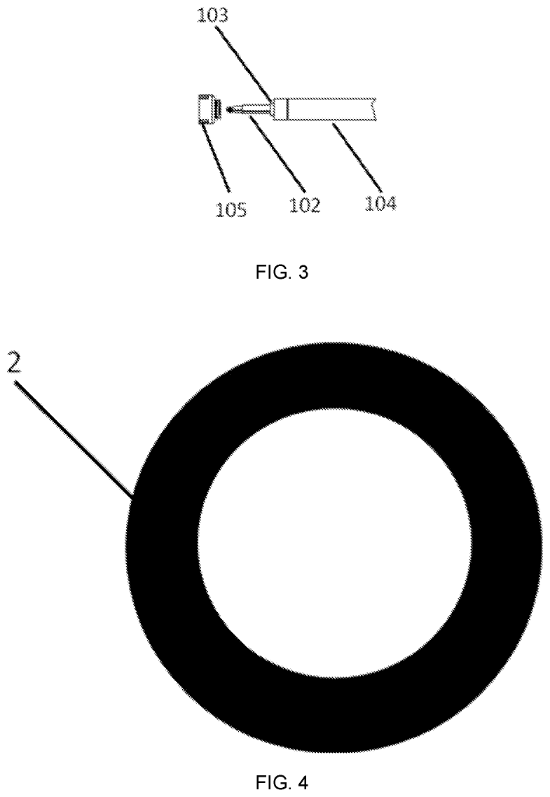

As shown in FIG. 3, the front end of the loading sleeve 104 is detachably connected to the tail part of the loading slot 105. Preferably, the front end of the loading sleeve 104 is provided with an internal thread, and the tail part of the loading slot 105 is provided with an external thread. The front end of the loading sleeve 104 and the tail part of the loading slot 105 are detachably connected with each other by threads.

As shown in FIG. 7, the daughter magnetic ring 2 is placed in the loading slot 105. Preferably, a snap ring is provided in the loading slot 105. The outer circumference of the daughter magnetic ring 2 is provided with a snap groove. The daughter magnetic ring 2 is loaded in the loading slot 105 by making the snap ring snapped in the snap groove.

In the present application, the loading slot 105 has a variety of specifications, so as to achieve cooperation with the daughter magnetic rings 2 of different diameters. Since the loading slot 105 and the loading sleeve 104 are detachably connected to each other, the daughter magnetic ring 2 of an appropriate specification can be selected according to the actual situation in the operation, and one design of an emission gun can meet requirements of a variety of application scenarios, improving the accuracy of the operation and reducing the cost of patients at the same time.

Preferably, the emission gun 1 further comprises a display pin 111 and a display frame 112, wherein the display pin 111 is fixed on the butt-joint rod 102 so that it can move along with the forward and backward movements of the butt-joint rod 102. The display frame 112 is arranged in the housing 108 and is used to display the position of the display pin 111. In this way, the display pin 111 and the display frame 112 are in combination, which can display the relative positions of the daughter magnetic ring 2 and the parent magnetic ring 3, and can assist the excitation operation, which improves the safety of the laparoscopic magnetic compression cholangioenterostomy.

More preferably, the emission gun 1 further comprises a safety bolt 106, wherein the activation sleeve 103 is connected to the safety bolt 106. The safety bolt 106 can restrict the movement of the activation mechanism 107, avoiding the accidental excitation of the daughter magnetic ring, reducing the occurrence of adverse events, and further improving the safety of the laparoscopic magnetic compression cholangioenterostomy.

More preferably, the push rotary button 101, the loading slot 105, the safety bolt 106, the activation mechanism 107, the housing 108, the sensory feedback mechanism 109, the display pin 111 and the display frame 112 are made of polymer material. The butt-joint rod 102, the activation sleeve 103, the loading sleeve 104 and the striker 110 are made of non-paramagnetic stainless steel material. In this way, they will not affect the magnetic properties of the daughter magnetic ring 2 and the parent magnetic ring 3 and the mutual attraction between the daughter magnetic ring 2 and the parent magnetic ring 3, and meet the medical and health requirements.

As shown in FIG. 1, when in use, the emission gun 1 can be sterilized by means of ethylene oxide. The gun head portion is inserted into the abdominal cavity through the laparoscope, by poking and snapping, and enters the intestinal cavity through the stump of the intestinal tube C. As shown in FIG. 7, the loading slot 105 is composed of a magnetic ring slot on the head and a positioning thread on the tail, and the daughter magnetic ring 2 can be placed in the magnetic ring slot. The magnetic ring slot has a variety of specifications, and the selection is made with reference to the size of the daughter magnetic ring 2, according to the actual situation in the operation. After the daughter magnetic ring 2 and the loading slot 105 are assembled, the assembly is completed by combining the positioning thread at the tail of the loading slot 105 and the internal thread at the front end of the loading sleeve 104. The combination of the loading slot 105 and the loading sleeve 104 is of a detachable design, which facilitates the selection and replacement of the daughter magnetic ring 2.

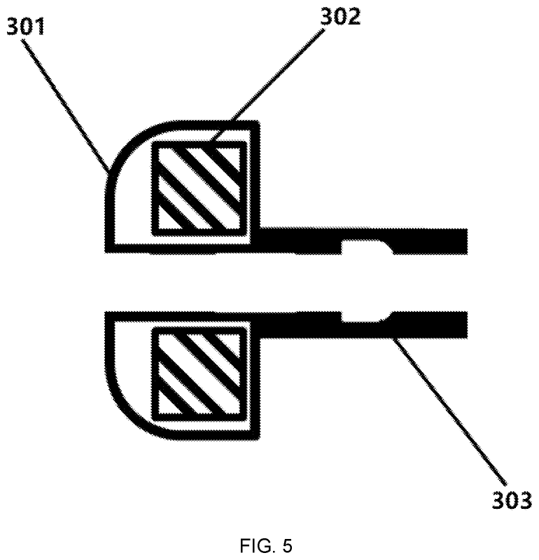

The push rotary button 101 is turned clockwise to move the butt-joint rod 102 forward (away from the daughter magnetic ring 2). At this time, the display pin 111 on the butt-joint rod 102 also moves forward, and its relative position is displayed by the display frame 112. As shown in FIGS. 5 and 8, a slot structure is provided in the inner drainage tube 303 of the parent magnetic ring 3, the slot structure capable of being in butt joint with the hook structure at the front end of the butt-joint rod 102 to realize the connection between the parent magnetic ring 3 and the emission gun 1. It should be noted that FIG. 8 is merely a schematic diagram for the purpose of showing the butt joint between the parent magnetic ring 3 and the butt-joint rod 102.

After the hook structure at the front end of the butt-joint rod 102 and the slot structure of the parent magnetic ring 3 are in butt joint, the position of the display pin 111 displayed on the display frame 112 is the relative position of the parent magnetic ring 3 and the daughter magnetic ring 2. At this time, the push rotary button 101 is turned counterclockwise to make the butt-joint rod 102 move backward (toward the daughter magnetic ring 2). At the same time, the butt-joint rod 102 drives the parent magnetic ring 3 to move backward (toward the daughter magnetic ring 2). When the display pin 111 reaches the excitation area in the display frame 112, it is prompted that the relative positions of the daughter magnetic ring 2 and the parent magnetic ring 3 are appropriate, and the mutual attraction has been achieved, and the daughter magnetic ring 2 can be launched.

Then, the safety bolt 106 is pulled down, and its restriction on the movement of the activation mechanism 107 is released, and the activation mechanism 107 is activated (pulled). At this time, under the action of the activation mechanism 107, the activation sleeve 103 moves forward. As shown in FIG. 9, when the activation sleeve 103 reaches the functional position, it can push the daughter magnetic ring 2 to move forward, so as to escape from the loading slot 105 to realize the separation of the daughter magnetic ring 2 and the emission gun 1. At the same time, the butt-joint rod 102 remains different under the action of inertia, and the parent magnetic ring 3 and the daughter magnetic ring 2 move forward together under the action of the activation sleeve 103, enabling occurrence of backward relative movement of the butt-joint rod 102 and the parent magnetic ring 3, so that the hook structure at the front end of the butt-joint rod 102 is disengaged from the slot structure in the inner drainage tube 303, and that the parent magnetic ring 3 and the emission gun 1 are separated from each other, to complete the biliary-intestinal anastomosis. At this time, the sensory feedback mechanism 109 on the activation sleeve 103 will be in contact with the striker 110, generating a “click” sound and making the operator feel a sense of breakthrough. After the excitation action is completed, the emission gun 1 is withdrawn from the human body. It should be noted that FIG. 9 is merely to show that the activation sleeve pushes the daughter magnetic ring to be disengaged from the loading slot and separates the parent magnetic ring from the emission gun. It is not the actual structural diagram of the magnetic compression anastomosis system for the laparoscopic cholangioenterostomy of the present application. In addition, in FIG. 9, for simplification, the snap ring of the loading slot 105 and the snap groove of the daughter magnetic ring 2 are not shown.

As shown in FIG. 4, in the present application, the daughter magnetic ring 2 is ring-shaped, made of N50 neodymium iron boron material, and coated with titanium nitride on the surface, with an inner diameter of 5 mm and an outer diameter of 8-15 mm. Because the outer diameter has different specifications (8-15 mm), it can meet the requirements of different application conditions. The daughter magnetic ring 2 with different outer diameters (8-15 mm) can be selected according to the actual width of the bile duct during the operation, so as to avoid biliary stricture caused by the too small diameter, and the bile leakage caused by the too large diameter.

When in use, according to the requirements in the operation, a daughter magnetic ring 2 of a suitable size is selected to be loaded in the loading slot 105.

As shown in FIG. 5, in the present application, the parent magnetic ring 3 is of a hollow mushroom head shape, which is composed of a parent magnetic casing 301, a parent magnet 302 and an inner drainage tube 303. The parent magnet 302 is loaded in the parent magnetic casing 301. The parent magnetic casing 301 is fixedly connected to the inner drainage tube 303. Preferably, the parent magnetic casing 301 and the inner drainage tube 303 are connected with each other by laser welding. The inner drainage tube 303 is provided with a slot structure therein. As shown in FIG. 8, the slot structure can be in butt joint with the hook structure at the front end of the butt-joint rod 102.

Preferably, both the parent magnetic casing 301 and the inner drainage tube 303 are made of non-paramagnetic stainless steel material and have an inner diameter of 3 mm. The outer diameter of the parent magnetic casing 301 is 8-15 mm. The outer diameter of the inner drainage tube 303 is 4 mm. The parent magnet 302 is ring-shaped, made of N50 neodymium iron boron material, and coated with titanium nitride on the surface, with an inner diameter of 5 mm and an outer diameter of 7.5 to 14.5 mm. Since the outer diameter of the parent magnetic ring 3 has different specifications (8-15 mm), it can meet the requirements of different application conditions.

When in use, the parent magnetic ring 3 is sterilized by means of ethylene oxide. According to the actual width of the bile duct during the operation, different outer diameters (8-15 mm) are selected, and it enters the abdominal cavity by poking and snapping, and placed at the tail end of the bile duct by means of purse string suture manner. The inner drainage tube 303 cooperates with the assisting rod 4 so that the inner drainage tube 303 can be in butt joint with the butt-joint rod 102 to realize the combination of the parent magnetic ring 3 and the emission gun 1. After the emission gun 1 is excited, the parent magnetic ring 3 is coupled with the daughter magnetic ring 2 through the parent magnet 302 to complete the intestinal anastomosis. At this time, the inner drainage tube 303 can immediately introduce the bile into the intestinal tube.

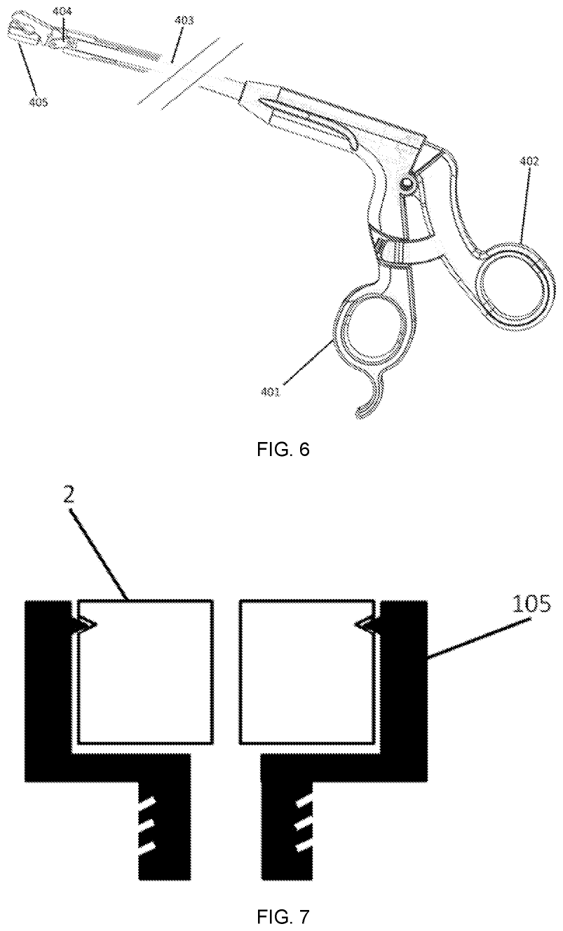

As shown in FIG. 6, in the present application, the assisting rod 4 is of a gun-like design, which is composed of a supporting mechanism 401, a triggering mechanism 402, a connecting rod 403, a movable joint 404 and a clamping mechanism 405, wherein the supporting mechanism 401 provides support for the triggering mechanism 402 and the clamping mechanism 405. The triggering mechanism 402 is connected to the supporting mechanism 401 through a first rotation shaft, so that the triggering mechanism 402 can be opened and closed relative to the supporting mechanism 401, around the first rotation shaft. The rear end of the connecting rod 403 is connected to the triggering mechanism 402, and the front end thereof is connected to the rear end of the movable joint 404 through a second rotating shaft. The front end of the movable joint 404 is connected to the clamping mechanism 405 through a third rotating shaft. In this way, when the triggering mechanism 402 expands relative to the supporting mechanism 401 around the first rotating shaft, it will drive the connecting rod 403 to move in a direction toward the clamping mechanism 405, and the opening of the clamping mechanism 405 is achieved by mans of the movable joint 404. Correspondingly, when the triggering mechanism 402 is closed relative to the supporting mechanism 401, around the first rotating shaft, it will drive the connecting rod 403 to move in a direction toward the supporting mechanism 401, and the closing of the clamping mechanism 405 is realized by the movable joint 404. The clamping mechanism 405 can clamp the inner drainage tube 303 of the parent magnetic ring 3, thereby fixing the parent magnetic ring 3 at the relative position of the stump of the common bile duct B, and assisting the parent magnetic ring 3 to be in butt joint with the emission gun 1.

Preferably, the supporting mechanism 401 and the triggering mechanism 402 are made of polymer material. The connecting rod 403, the movable joint 404 and the clamping mechanism 405 are made of non-paramagnetic stainless steel. In this way, they will not affect the magnetic properties of the daughter magnetic ring 2 and the parent magnetic ring 3 and the mutual attraction between the daughter magnetic ring 2 and the parent magnetic ring 3, and meet the medical and health requirements.

When in use, the assisting rod 4 is sterilized by means of ethylene oxide, and the front end part can enter, by poking and snapping, the abdominal cavity through a laparoscope. The clamping mechanism 405 can be opened and closed by operating the triggering mechanism 402. During the operation, the inner drainage tube 303 is clamped by the clamping mechanism 405, so that the relative position of the parent magnetic ring 3 can be fixed, and it will not be retracted into the bile duct due to operational factors (the purse string suture needs to be performed again at this time). In addition, it is also helpful to realize the butt joint of the parent magnetic ring 3 and the emission gun 1. After the butt joint of the parent magnetic ring 3 and the emission gun 1 is complete, the assisting rod 4 is withdrawn from the human body.

The working principle of the present application will be described in detail below.

As shown in FIG. 1, when performing a laparoscopic cholangioenterostomy operation, firstly, the specifications of the daughter magnetic ring 2 and the parent magnetic ring 3 are selected according to the actual situation during the operation. The specification of the loading slot 105 is selected with reference to the size of the daughter magnetic ring 2, the daughter magnetic ring 2 is placed in the loading slot 105, and then the loading slot 105 is connected with the loading sleeve 104 to complete the assembly. A laparoscopic separation forceps is used to clamp the inner drainage tube 303 of the parent magnetic ring 3, and it is placed, by poking and snapping, into the stump of bile duct of the abdominal cavity through laparoscopy, and the purse string suture is performed, the parent magnetic ring 3 is placed in the bile duct, and then the laparoscopic separation forceps is removed, and the assisting rod 4 is placed therein. The inner drainage tube 303 is clamped by the clamping mechanism 405 to fix the parent magnetic ring 3 at the relative position in the abdominal cavity. The emission gun 1, which is loaded with the daughter magnetic ring 2, enters, by poking and snapping, the abdominal cavity through the laparoscope, and then enters the intestinal stump. After reaching the predetermined position, the push rotary button 101 is turned clockwise to move the butt-joint rod 102 forward (away from the daughter magnetic ring 2). The head end of the butt-joint rod 102 breaks through the wall of the intestinal tube, and the assisting rod 4 is operated to make the parent magnetic ring 3 and the emission gun 1 move toward each other, so that the slot structure of the inner drainage tube 303 can be in butt joint with the butt-joint rod 102 using the hook structure at the front end. At this time, the butt joint between the parent magnetic ring 3 and the emission gun 1 is completed. The assisting rod 4 is removed and the push rotary button 101 is turned counterclockwise. When the display pin 111 reaches the excitation area in the display frame 112, it will be prompted that the relative positions of the daughter magnetic ring 2 and the parent magnetic ring 3 are appropriate, and mutual attraction has been achieved, and the launch operation of the daughter magnetic ring 2 can be performed. At this time, the safety bolt 106 is pulled down and the activation mechanism 107 is activated. When it is heard that the sensory feedback mechanism 109 contacts the striker 110 which makes a “click” sound, and a sense of breakthrough is felt, it is prompted that the coupling of the daughter magnetic ring 2 and the parent magnetic ring 3 is completed and the daughter magnetic ring 2 has been launched, and the emission gun 1 is removed and the operation is completed.

The combination of the loading slot 105 and the loading sleeve 104 is of a detachable design, which allows that the daughter magnetic ring 2 of an appropriate specification is selected according to the actual situation in the operation. One design of the emission gun can meet the requirements of multiple application scenarios, improving the accuracy of operation, while reducing the cost of patients. The design of the clamping mechanism 405 being in the assisting rod 4 can effectively fix the relative position of the parent magnetic ring 3, so as to prevent it from retracting into the bile duct, avoiding performing the purse string sutures again, and easily realize the butt joint with the emission gun 1. The display pin 111 is combined with the display frame 112 to display the relative positions of the daughter magnetic ring 2 and the parent magnetic ring 3, and can assist the excitation operation. The sensory feedback mechanism 109 will cooperate with the striker 110 to generating a “click” sound and making the operator feel a sense of breakthrough, which can give a feedback on the completion of the exciting action. The safety bolt 106 can restrict the movement of the activation mechanism 107, avoiding the accidental excitation of the daughter magnetic ring 2 and reducing the occurrence of adverse events.

The magnetic compression anastomosis system for laparoscopic cholangioenterostomy of the present application can not only greatly improve the accuracy of laparoscopic magnetic compression cholangioenterostomy, and reduce the incidence of biliary stricture and bile leakage, but also reduce the difficulty of performing the laparoscopic magnetic compression cholangioenterostomy, shorten the operation time and reduce the learning curve. In addition, through the addition of the assisting structures, it also greatly enhances the safety of the laparoscopic magnetic compression cholangioenterostomy.

Those skilled in the art can understand that although some embodiments described herein comprise certain features, but not all, of the features included in other embodiments, the combination of features of different embodiments means that they fall within the scope of the present application, and form different embodiments.

Although the embodiments of the present application have been described with reference to the drawings, those skilled in the art can make various modifications and variations without departing from the spirit and scope of the present application, and such modifications and variations all fall within the scope defined by the claims herewith.

1. A magnetic compression anastomosis system for laparoscopic cholangioenterostomy, comprising an emission gun, a daughter magnetic ring, a parent magnetic ring and an assisting rod, wherein the parent magnetic ring is positioned at a stump of a common bile duct in a purse string suture manner, and a relative position thereof is fixed by the assisting rod, the emission gun is loaded with the daughter magnetic ring and configured to make the daughter magnetic ring be in butt joint with the parent magnetic ring by an intestinal cavity channel of an intestine tube, and the assisting rod is configured to assist the parent magnetic ring to be in butt joint with the emission gun, wherein a distance between the daughter magnetic ring and the parent magnetic ring is continuously shortened by a push rotary button of the emission gun, wherein when a relative distance between the daughter magnetic ring and the parent magnetic ring is shortest and mutual attraction is achieved, separation of the daughter magnetic ring from the emission gun is achieved by an activation mechanism of the emission gun, achieving laparoscopic cholangioenterostomy.

2. The magnetic compression anastomosis system according to claim 1, wherein the emission gun comprises a push rotary button, a butt-joint rod, an activation sleeve, a loading sleeve, a loading slot, the activation mechanism, a housing, a sensory feedback mechanism and a striker, wherein a front end of the push rotary button is connected to a rear end of the butt-joint rod through a spring mechanism, a front end of the butt-joint rod is provided with a hook structure configured to be able to be in butt joint with the parent magnetic ring, the butt-joint rod is located in the activation sleeve, the activation sleeve is connected to the activation mechanism, the sensory feedback mechanism is fixed on the activation sleeve, and the striker is arranged in the housing, wherein when the activation sleeve moves forward, the sensory feedback mechanism can contact the striker, the activation sleeve is located in the loading sleeve, the loading sleeve is fixed on the housing, a front end of the loading sleeve is detachably connected to a tail part of the loading slot, and the daughter magnetic ring is placed in the loading slot.

3. The magnetic compression anastomosis system according to claim 2, wherein the emission gun further comprises a display pin and a display frame, the display pin is fixed on the butt-joint rod, and the display frame is arranged in the housing and configured to display a position of the display pin.

4. The magnetic compression anastomosis system according to claim 3, wherein the emission gun further comprises a safety bolt, and the activation sleeve is connected to the safety bolt.

5. The magnetic compression anastomosis system according to claim 4, wherein the push rotary button, the loading slot, the safety bolt, the activation mechanism, the housing, the sensory feedback mechanism, the display pin and the display frame are made of a polymer material, and the butt-joint rod, the activation sleeve, the loading sleeve and the striker are made of a non-paramagnetic stainless steel material.

6. The magnetic compression anastomosis system according to claim 1, wherein the daughter magnetic ring is ring-shaped, made of N50 neodymium iron boron (NdFeB) material, and coated with titanium nitride (TiN) on a surface thereof, and has an inner diameter of 5 mm and an outer diameter of 8-15 mm.

7. The magnetic compression anastomosis system according to claim 2, wherein the parent magnetic ring is composed of a parent magnetic casing, a parent magnet and an inner drainage tube, wherein the parent magnet is loaded in the parent magnet casing, the parent magnet casing is fixedly connected with the inner drainage tube, and the inner drainage tube is provided therein with a slot structure, and the slot structure is configured to be able to be in butt joint with the hook structure at the front end of the butt-joint rod.

8. The magnetic compression anastomosis system according to claim 7, wherein both the parent magnetic casing and the inner drainage tube are made of a non-paramagnetic stainless steel material and have an inner diameter of 3 mm, an outer diameter of the parent magnetic casing is 8-15 mm, an outer diameter of the inner drainage tube is 4 mm, and the parent magnet is ring-shaped and made of N50 neodymium iron boron material, and is coated with titanium nitride on a surface thereof, and has an inner diameter of 5 mm and an outer diameter of 7.5-14.5 mm.

9. The magnetic compression anastomosis system according to claim 7, wherein the assisting rod consists of a supporting mechanism, a triggering mechanism, a connecting rod, a movable joint, and a clamping mechanism, wherein the supporting mechanism provides support for the triggering mechanism and the clamping mechanism, and the triggering mechanism is connected to the supporting mechanism through a first rotating shaft, so that the triggering mechanism can be opened and closed relative to the supporting mechanism, around the first rotating shaft, and a rear end of the connecting rod is connected to the triggering mechanism and a front end thereof is connected to a rear end of the movable joint through a second rotating shaft, and a front end of the movable joint is connected to the clamping mechanism through a third rotating shaft, wherein the triggering mechanism drives, when being opened relative to the supporting mechanism around the first rotating shaft, the connecting rod to move in a direction toward the clamping mechanism and realizes through the movable joint opening of the clamping mechanism, and correspondingly, the triggering mechanism drives, when being closed relative to the supporting mechanism around the first rotating shaft, the connecting rod to move in a direction toward the supporting mechanism, and realize closing of the clamping mechanism through the movable joint, and the clamping mechanism is configured to clamp the inner drainage tube of the parent magnetic ring, thereby fixing the parent magnetic ring at a relative position of the stump of the common bile duct and assisting the parent magnetic ring to be in butt joint with the emission gun.

10. The magnetic compression anastomosis system according to claim 9, wherein the supporting mechanism and the triggering mechanism are made of a polymer material, and the connecting rod, the movable joint and the clamping mechanism are made of a non-paramagnetic stainless steel.
